SEQUENCE LISTING (2 sequences)

SEQ ID NO: 1 - RNA, 48 nt, Homo sapiens
  source 1..48: mol_type = mRNA
ccacaaagagtctacatgtctagggtctagacatgttcagctttgtgg

SEQ ID NO: 2 - RNA, 46 nt, Homo sapiens
  source 1..46: mol_type = mRNA
cacaaggagtctgcatgtctaagtgctagacatgctcagctttgtg

(19) United States
(12) Patent Application Publication
(10) Pub. No.: US 2023/0313296 A1
(43) Pub. Date: Oct. 5, 2023

(54) USING FLUORESCENT COLLAGEN .alpha. 1(I) AND .alpha. 2(I) mRNAs TO SCREEN DRUG CANDIDATES

(71) Applicant: Florida State University Research Foundation, Inc., Tallahassee, FL (US)

(72) Inventor: Branko Stefanovic, Tallahassee, FL (US)

(73) Assignee: Florida State University Research Foundation, Inc., Tallahassee, FL (US)

(21) Appl. No.: 18/122,289

(22) Filed: Mar. 16, 2023

Related U.S. Application Data
(60) Provisional application No. 63/320,779, filed on Mar. 17, 2022.

(51) Int. Cl. G01N 33/58; C12Q 1/6876

(52) U.S. Cl. CPC C12Q 1/6876; G01N 33/582; C12Q 2600/136

(57) ABSTRACT

Fluorescent collagen a1(I) and a2(I) mRNA compounds can be used to detect whether a test compound affects the binding of collagen a1(I) and/or a2(I) mRNAs to LARP6 protein. Fluorescent collagen a1(I) and a2(I) mRNA compounds include collagen a1(I) and a2(I) mRNA sequences that have been tagged with a fluorophore and have LARP6 binding affinity.

### CROSS-REFERENCE TO RELATED APPLICATION

This claims the benefit of priority to Application No. 63/320,779, entitled “USING FLUORESCENT COLLAGEN a1(I) and a2(I) mRNAs TO SCREEN DRUG CANDIDATES,” filed Mar. 17, 2022, which is incorporated by reference in its entirety.

### FIELD

This relates to the field of drug screening and, more particularly, to using fluorescence techniques to screen drug candidates for antifibrotic activity.

### SEQUENCE LISTING

The application contains a Sequence Listing electronically submitted via EFS-web to the United States Patent and Trademark Office as an xml file named “Sequence_Listing.xml,” which was created Mar. 9, 2023 and is 3 kB. The electronically filed Sequence Listing serves as both the paper copy required by 37 C.F.R. § 1.821(c) and the computer readable file required by 37 C.F.R. § 1.821(c). The information contained in the Sequence Listing is incorporated by reference herein in its entirety

### BACKGROUND

Type I collagen is the most abundant protein in vertebrates. It makes up >90% of the organic mass of the bone and is the main constituent of tendon, skin, ligaments, cornea, arterial blood vessel walls, and many interstitial connective tissues.

Type I collagen is composed of two a1(I) and one a2(I) polypeptide chains that are encoded by two mRNAs, namely, the collagen a1(I) and a2(I) mRNAs. After assembly, triple helices of type I procollagen are secreted into the extracellular space, where the globular domains are cleaved off and the triple helices are polymerized into fibrils.

The collagen a1(I) and a2(I) mRNAs have in their 5′UTR a unique sequence, termed the collagen 5′ stem-loop (5′SL). The 5′SL of the a1(I) and a2(I) mRNAs have an active fold that binds to La-related protein 6 or “LARP6.” Pat. No. 8,697,385 reports that the LARP6 is specifically involved in Type I collagen regulation.

### BRIEF SUMMARY

Fluorescent RNA sensors that can be used to detect whether a test compound affects the binding of collagen a1(I) and a1(I) mRNAs to LARP6 protein are described. The fluorescent RNA sensors include collagen a1(I) and a2(I) mRNA sequences that have been tagged with a fluorophore.

A first example of a composition comprises a first collagen mRNA nucleotide sequence having at least 90% sequence identity with SEQ ID NO: 1 and a binding affinity for LARP6. The first collagen mRNA nucleotide sequence has at least one fluorophore located at nucleic acid position G10 and/or C38 of SEQ ID NO: 1.

This first composition may include one or more of the following additional features.

The first collagen mRNA nucleotide sequence may be SEQ ID NO: 1 having at least one fluorophore located at nucleic acid position G10 and/or C38 of SEQ ID NO: 1.

The at least one fluorophore located at nucleic acid position G10 and/or C38 of SEQ ID NO: 1 may be substituted at position G10 and/or C38 of SEQ ID NO: 1.

The at least one fluorophore located at nucleic acid position G10 and/or C38 of SEQ ID NO: 1 may include 2-aminopurine substituted at nucleic acid position G10 and/or pyrrolo-cytidine substituted at nucleic acid position C38.

The first collagen mRNA nucleotide sequence may be SEQ ID NO: 1 having nucleic acid position G10 substituted for 2-aminopurine.

The first composition may further comprise a second collagen mRNA nucleotide sequence having at least 90% sequence identity with SEQ ID NO: 2 and a binding affinity for LARP6. The second collagen mRNA nucleotide sequence may have at least one fluorophore located at nucleic acid position G9 and/or C37 of SEQ ID NO: 2.

The second collagen mRNA nucleotide sequence may be SEQ ID NO: 2 having at least one fluorophore located at nucleic acid position G9 and/or C37 of SEQ ID NO: 2.

The at least one fluorophore located at nucleic acid position G9 and/or C37 of SEQ ID NO: 2 may be substituted at position G9 and/or C37 of SEQ ID NO: 2.

The at least one fluorophore located at nucleic acid position G9 and/or C37 of SEQ ID NO: 2 may include 2-aminopurine substituted at nucleic acid position G9 and/or pyrrolo-cytidine substituted at nucleic acid position C37.

The second collagen mRNA nucleotide sequence may be SEQ ID NO: 2 having nucleic acid position C37 substituted for pyrrolo-cytidine.

An example of a second composition comprises a first collagen mRNA nucleotide sequence having at least 90% sequence identity with SEQ ID NO: 2 and a binding affinity for LARP6, the first collagen mRNA nucleotide sequence having at least one fluorophore located at nucleic acid position G9 and/or C37 of SEQ ID NO: 2.

This second composition may include one or more of the following additional features.

The first collagen mRNA nucleotide sequence may be SEQ ID NO: 2 having at least one fluorophore located at nucleic acid position G9 and/or C37 of SEQ ID NO: 2.

The at least one fluorophore located at nucleic acid position G9 and/or C37 of SEQ ID NO: 2 may be substituted at position G9 and/or C37 of SEQ ID NO: 2.

The at least one fluorophore located at nucleic acid position nucleic acid position G9 and/or C37 of SEQ ID NO: 2 may include 2-aminopurine substituted at nucleic acid position G9 and/or pyrrolo-cytidine substituted at nucleic acid position C37.

The first collagen mRNA nucleotide sequence may be SEQ ID NO: 2 having nucleic acid position C37 substituted for pyrrolo cytidine.

The second composition may further comprise a second collagen mRNA nucleotide sequence having at least 90% sequence identity with SEQ ID NO: 1 and a binding affinity for LARP6. The second collagen mRNA nucleotide sequence may have at least one fluorophore located at nucleic acid position G10 and/or C38 of SEQ ID NO: 1.

The second collagen mRNA nucleotide sequence may be SEQ ID NO: 1 having at least one fluorophore located at nucleic acid position G10 and/or C38 of SEQ ID NO: 1.

The at least one fluorophore located at nucleic acid position G10 and/or C38 of SEQ ID NO: 1 may be substituted at position G10 and/or C38 of SEQ ID NO: 1.

The at least one fluorophore located at nucleic acid position G10 and/or C38 of SEQ ID NO: 1 may include 2-aminopurine substituted at nucleic acid position G10 and/or pyrrolo-cytidine substituted at nucleic acid position C38.

The second collagen mRNA nucleotide sequence may be SEQ ID NO: 1 having nucleic acid position G10 substituted for 2-aminopurine.

An example of a first method comprises detecting whether a test compound affects binding of a fluorescent RNA sensor to LARP6 by contacting the fluorescent RNA sensor with the test compound. The fluorescent RNA sensor comprises a first collagen mRNA nucleotide sequence having at least 90% sequence identity with SEQ ID NO: 1 and a binding affinity for LARP6, the first collagen mRNA nucleotide sequence having at least one fluorophore located at nucleic acid position G10 and/or C38 of SEQ ID NO: 1.

This first method may include one or more of the following additional features.

The first collagen mRNA nucleotide sequence may be SEQ ID NO: 1 having at least one fluorophore located at nucleic acid position G10 and/or C38 of SEQ ID NO: 1.

The at least one fluorophore located at nucleic acid position G10 and/or C38 of SEQ ID NO: 1 may be substituted at position G10 and/or C38 of SEQ ID NO: 1.

The at least one fluorophore located at nucleic acid position G10 and/or C38 of SEQ ID NO: 1 may include 2-aminopurine substituted at nucleic acid position G10 and/or pyrrolo-cytidine substituted at nucleic acid position C38.

The first collagen mRNA nucleotide sequence is SEQ ID NO: 1 having nucleic acid position G10 substituted for 2-aminopurine.

The fluorescent RNA sensor may further comprise a second collagen mRNA nucleotide sequence having at least 90% sequence identity with SEQ ID NO: 2 and a binding affinity for LARP6, the second collagen mRNA nucleotide sequence having at least one fluorophore located at nucleic acid position G9 and/or C37 of SEQ ID NO: 2.

The second collagen mRNA nucleotide sequence may be SEQ ID NO: 2 having at least one fluorophore located at nucleic acid position G9 and/or C37 of SEQ ID NO: 2.

The at least one fluorophore located at nucleic acid position G9 and/or C37 of SEQ ID NO: 2 may be substituted at position G9 and/or C37 of SEQ ID NO: 2.

The at least one fluorophore located at nucleic acid position G9 and/or C37 of SEQ ID NO: 2 may include 2-aminopurine substituted at nucleic acid position G9 and/or pyrrolo-cytidine substituted at nucleic acid position C37.

The second collagen mRNA nucleotide sequence may be SEQ ID NO: 2 having nucleic acid position C37 substituted for pyrrolo-cytidine.

A second example of a method comprises detecting whether a test compound affects binding of a fluorescent RNA sensor to LARP6 by contacting the fluorescent RNA sensor with the test compound. The fluorescent RNA sensor includes a first collagen mRNA nucleotide sequence having at least 90% sequence identity with SEQ ID NO: 2 and a binding affinity for LARP6, the first collagen mRNA nucleotide sequence having at least one fluorophore located at nucleic acid position G9 and/or C37 of SEQ ID NO: 2.

This second method may include one or more of the following additional features.

The first collagen mRNA nucleotide sequence may be SEQ ID NO: 2 having at least one fluorophore located at nucleic acid position G9 and/or C37 of SEQ ID NO: 2.

The at least one fluorophore located at nucleic acid position G9 and/or C37 of SEQ ID NO: 2 may be substituted at position G9 and/or C37 of SEQ ID NO: 2.

The at least one fluorophore located at nucleic acid position G9 and/or C37 of SEQ ID NO: 2 may include 2-aminopurine substituted at nucleic acid position G9 and/or pyrrolo-cytidine substituted at nucleic acid position C37.

The first collagen mRNA nucleotide sequence may be SEQ ID NO: 2 having nucleic acid position C37 substituted for pyrrolo cytidine.

The second method may further comprise a second collagen mRNA nucleotide sequence having at least 90% sequence identity with SEQ ID NO: 1 and a binding affinity for LARP6. The second collagen mRNA nucleotide sequence may have at least one fluorophore located at nucleic acid position G10 and/or C38 of SEQ ID NO: 1.

The second collagen mRNA nucleotide sequence may be SEQ ID NO: 1 having at least one fluorophore located at nucleic acid position G10 and/or C38 of SEQ ID NO: 1.

The at least one fluorophore located at nucleic acid position G10 and/or C38 of SEQ ID NO: 1 may be substituted at position G10 and/or C38 of SEQ ID NO: 1.

The at least one fluorophore located at nucleic acid position G10 and/or C38 of SEQ ID NO: 1 may include 2-aminopurine substituted at nucleic acid position G10 and/or pyrrolo-cytidine substituted at nucleic acid position C38.

The second collagen mRNA nucleotide sequence may SEQ ID NO: 1 having nucleic acid position G10 substituted for 2-aminopurine.

The compositions and methods may include any combination of the aforementioned features.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a diagram of the sequence of the 5′stem-loop element of type I collagen mRNAs. SEQ ID NO: 1, on the left, is the sequence of the 5′ stem loop of human collagen a1(I) mRNA. SEQ ID NO: 2, on the right, is the sequence of the 5′ stem loop of human collagen a2(I) mRNA. The nucleotides associated with LARP6 binding are circled. The translation start codons are boxed. The LARP6 binding site is circled in dashed lines. Examples of possible fluorophore locations are indicated by arrows.

FIG. 2 is a graph of the increase in fluorescence of the fluorescent collagen a1(I) 5′SL compound with increasing concentration of LARP6. The arrow indicates the increase in intensity with increasing concentration of the LA-domain. Excitation spectra are to the left, emission spectra are to the right.

FIG. 3 is a bar graph of the quantitative change of fluorescent intensity of the fluorescent collagen a1(I) 5′SL compound upon binding of saturating amounts of the LA-domain.

FIG. 4 is a graph of the decrease in fluorescence of the fluorescent collagen a2(I) 5′SL compound with increasing concentration of the LA-domain. The arrow indicates the decrease in intensity with increasing concentration of LARP6. Excitation spectra are to the left, emission spectra are to the right.

FIG. 5 includes graphs of the response of the fluorescent mRNA compounds to being contacted with a test compound (drug). The upper panel is a graph of the decrease in fluorescence intensity of the fluorescent collagen a1(I) 5′SL compound with increasing concentrations of the test compound. The lower panel is a graph of the decrease in fluorescence intensity of the fluorescent collagen a2(I) 5′SL compound with increasing concentrations of the test compound.

FIG. 6 is a gel mobility shift assay showing free RNA (RNA) and complex of LA-domain bound to RNA (RNA/LA). Lane 1 contains RNA only. Lane 2 contains the RNA/LA complex in absence of the test compound. Lanes 3-5 show dissociation of RNA/LA complexes with the test compound.

FIG. 7 is the Sequence Listing in txt format.

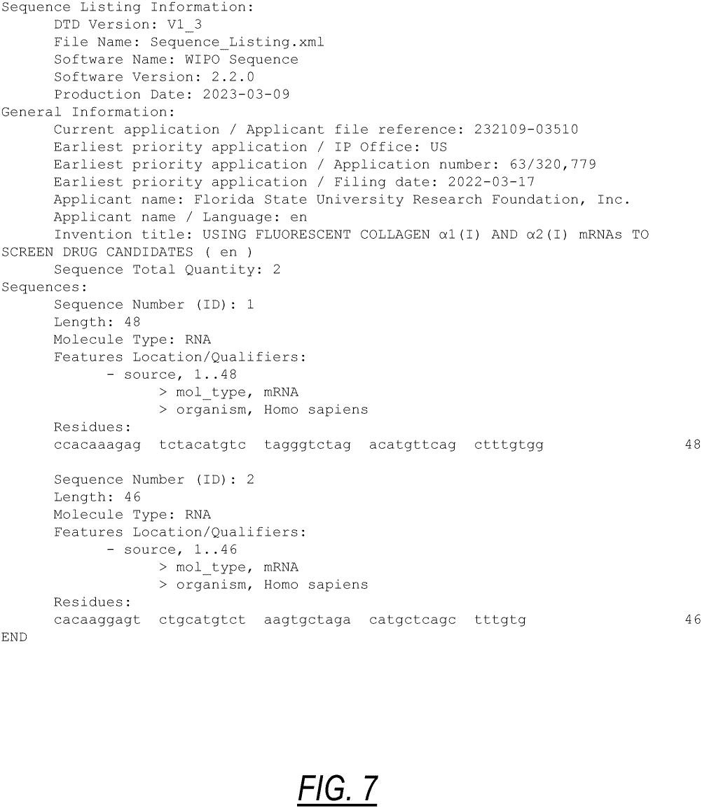

### DESCRIPTION

This disclosure describes examples of embodiments, but not all possible examples of the compositions and methods. Where a particular feature is disclosed in the context of a particular example, that feature can also be used, to the extent possible, in combination with and/or in the context of other features and examples. The compositions and methods may be embodied in many different forms and should not be construed as being limited to only the features and examples described here.

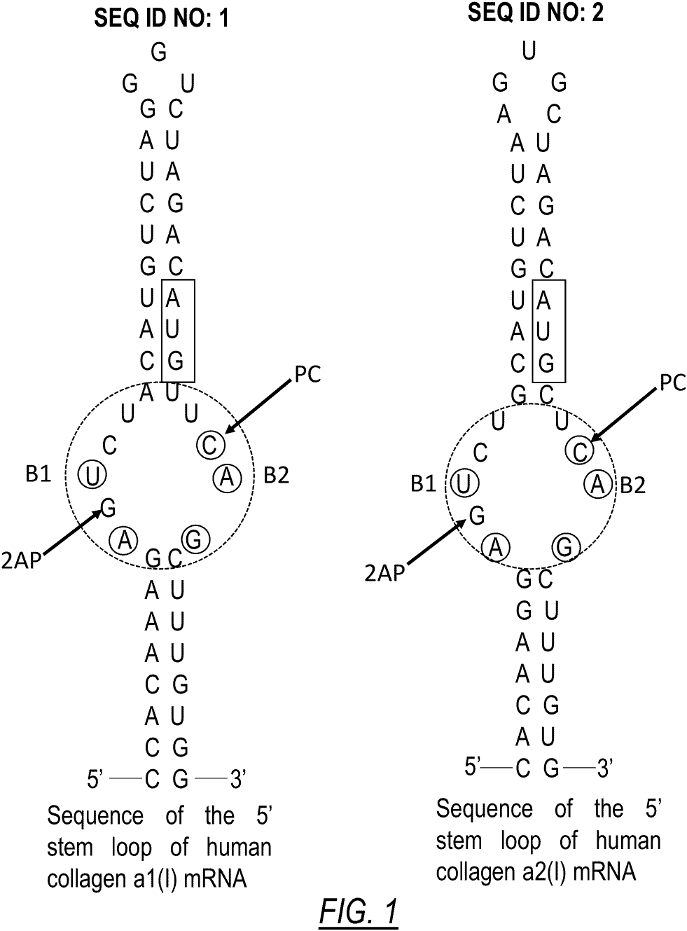

FIG. 1 is a diagram of the sequences of the collagen a1(I) and a2(I) 5′ stem loop human collagen mRNAs (SEQ ID NO: 1 and SEQ ID NO: 2) with the nucleotides involved in LARP6 binding circled and the stop codons boxed. Either or both of SEQ ID NO: 1 and SEQ ID NO: 2 may be modified to include a fluorophore to form a fluorescent collagen mRNA compound. The fluorescent collagen mRNA compound may be used to detect binding to LARP6 or binding to a test compound.

SEQ ID NO: 1

5' - CCACAAAGAGUCUACAUGUCUAGGGUCUAGACAUGUUCAGCUUUGUGG - 3'
5 10 15 20 25 30 35 40 45

SEQ ID NO: 2

5' - CACAAGGAGUCUGCAUGUCUAAGUGCUAGACAUGCUCAGCUUUGUG - 3'
5 10 15 20 25 30 35 40 45

The “U” symbols above represent uracil residues. In the Sequence Listing xml file the “U” symbols are replaced with “t” symbols to comply with Standard ST.26, which requires U residues in RNA sequences to be represented by the “t” symbol.

In SEQ ID NO: 1, the fluorophore may be located at position 10 or 38 (in bold) because the ring region is responsible for binding to LARP6.

In SEQ ID NO: 2, the fluorophore may be located at position 9 or 37 (in bold) because the ring region is responsible for binding to LARP6.

When LARP6 or another compound binds to fluorophore-labeled SEQ ID NO: 1 and/or 2, this changes the chemical environment of the fluorophore, which changes the fluorescence intensity compared to the fluorescence intensity of the fluorophore-labeled sequence that is not bound to LARP6 or a test compound.

These fluorescent collagen 5′ stem-loop mRNA compounds are useful in identifying antifibrotic compounds, such as drugs, because these sequences are unique to collagen mRNAs and regulate type I collagen expression by binding LARP6. Previous LARP6 inhibiting compounds targeted the protein, but antifibrotic compounds discovered with the technique disclosed here, which target the 5′ SL mRNA, may only affect the role of LARP6 in type I collagen biogenesis and not the other functions of LARP6. Such high specificity is important, because long term inhibition of LARP6 activity is useful to combat fibrosis.

There has previously been no method reported that is suitable for high-throughput drug screening to discover compounds targeting 5′SL mRNA sequences. The fluorescent collagen mRNA compounds described here are designed to detect interactions of molecules with the active site of the 5′ SL mRNAs and indicate the interaction as an increase or decrease in fluorescence intensity. Using both fluorescent collagen mRNA compounds can be used to cross-validate the results obtained by running tests with the individual fluorescent collagen mRNA compounds.

A first example of a composition useful for sensing 5′ SL mRNA binding includes a first collagen mRNA nucleotide sequence having at least 90% sequence identity with SEQ ID NO: 1 and a binding affinity for LARP6. The first collagen mRNA nucleotide sequence has at least one fluorophore located at nucleic acid position G10 and/or C38 of SEQ ID NO: 1.

Another example of a composition useful for sensing 5′ SL mRNA binding includes a second collagen mRNA nucleotide sequence having at least 90% sequence identity with SEQ ID NO: 2 and a binding affinity for LARP6. The second collagen mRNA nucleotide sequence has at least one fluorophore located at nucleic acid position G9 and/or C37 of SEQ ID NO: 2.

The first fluorescent compound and the second fluorescent compound have a binding affinity for LARP6. Having a binding affinity means the compound is capable of a binding interaction with LARP6.

The fluorophore on the first fluorescent compound is located at nucleic acid position 10 and/or 38 by being bound to an existing nucleic acid at this position or by being substituted at this position. This fluorophore is a fluorescent compound capable of being placed at the aforementioned position without substantially changing the structure of the mRNA to the extent the mRNA loses its LARP6 binding affinity.

In a particular example, a fluorophore on the first fluorescent compound is substituted at nucleic acid position 10 and/or 38. In this case, the fluorophore has a similar structure to the nucleic acid being substituted in order to avoid substantially changing the structure of the mRNA.

The fluorophore on the first fluorescent compound may, for example, be a fluorescent nucleic acid analog to the nucleic acid at position 10. 2-aminopurine (2AP), which is a purine analog of guanidine and adenine, is fluorescent and, therefore, is a possible example of this fluorophore. If 2-aminopurine is used as the fluorophore, it may be substituted for guanine at position G10, for example.

In another example, the fluorophore on the first fluorescent compound may, for example, a fluorescent nucleic acid analog to the nucleic acid at position 38. Pyrollo-cytidine (PC), which is a fluorescent nucleic acid analogue of cytidine, is a possible example of this fluorophore. If pyrollo-cytidine is used as the fluorophore, it may be substituted for cytidine at position C38, for example.

In another example, the first fluorescent compound may include 2AP at nucleic acid position 10 and/or PC at nucleic acid position 38.

The fluorophore on the second fluorescent compound is located at nucleic acid position 9 and/or 37 by being bound to an existing nucleic acid in one or more of these positions or by being substituted in one or more of these positions. This fluorophore is a fluorescent compound capable of being placed at the aforementioned positions without substantially changing the structure of the mRNA.

In a particular example, the fluorophore on the second fluorescent compound is substituted at position 9 and/or 37. In this case, the fluorophore has a similar structure to the nucleic acid being substituted in order to avoid substantially changing the structure of the mRNA.

The fluorophore on the second fluorescent compound may, for example, be a fluorescent nucleic acid analog to the nucleic acid at position 37. Pyrollo-cytidine is a possible example of the this fluorophore. If pyrollo-cytidine is used as the fluorophore, it may be substituted for cytidine at position C37, for example.

In another example, the fluorophore on the second fluorescent compound may, for example, be a fluorescent nucleic acid analog to the nucleic acid at position 9. 2-aminopurine is a possible example of the this fluorophore. If 2-aminopurine is used as this fluorophore, it may be substituted for guanidine at position G9, for example.

In another example, the second fluorescent compound may include 2AP at nucleic acid position 9 and/or PC at nucleic acid position 37.

In some examples, the fluorophore-labelled collagen a1(I) and a2(I) mRNAs have exact sequence identity with SEQ ID NO: 1 or SEQ ID NO: 2, respectively, except for cases in which the fluorophore is substituted for one or more nucleic acids in SEQ ID NO: 1 or SEQ ID NO: 2.

In other examples, the fluorophore-labelled collagen a1(I) and a2(I) mRNAs have at least 90% sequence identity with SEQ ID NO: 1 or SEQ ID NO: 2, respectively. In these examples certain nucleic acids in either sequence may be substituted for other nucleic acids with the proviso that the collagen mRNA nucleotide sequence having at least 90% sequence identity with SEQ ID NO: 1 or SEQ ID NO: 2 maintains its binding affinity for LARP6.

To allow for substitutions without destroying the collagen mRNA nucleotide sequence’s binding affinity for LARP6, substitutions in the 5′-stem loop region, other than to add a fluorophore as discussed above, should be avoided. In SEQ ID NO: 1 the 5′-stem loop region includes positions G8-A14 and U36-C41. In SEQ ID NO: 2 the 5′-stem loop region includes positions G7-G13 and C25-C30.

Either or both of the first and second fluorescent compounds may be used to determine whether a test compound can bind thereto. If the test compound binds to the first and/or second fluorescent compound, the fluorescence intensity of the first and/or second fluorescent compounds will change. By binding to the first and/or second fluorescent compound, the test compound may competitively inhibit or interfere with binding of to the collagen a1(I) 5′ stem loop mRNA and/or the collagen a2(I) 5′ stem loop mRNA, which would make the test compound a potential antifibrotic drug.

The test compound may be any compound being screened for its ability to competitively inhibit or interfere with binding of the collagen a1(I) 5′ stem loop mRNAs and/or the collagen a2(I) 5′ stem loop mRNA to LARP6. Using the first and/or second fluorescent compounds may provide a relatively fast method for screening many test compounds for their potential effectiveness as antifibrotic drugs.

An example of a screening method includes detecting whether the test compound affects binding of a fluorescent RNA sensor to LARP6 by contacting a fluorescent RNA sensor with the test compound. The fluorescent RNA sensor includes at least one of the first fluorescent compound including SEQ ID NO: 1 having a fluorophore located at nucleic acid position 10 and/or 38 and the second fluorescent compound including SEQ ID NO: 2 having a fluorophore located at nucleic acid position 9 and/or 37. The fluorescence of the fluorescent RNA sensor is detected to determine whether the test compound affects the fluorescence.

In this method, the test compound may affect the binding of the fluorescent RNA sensor by competitively inhibiting or interfering with binding of to the fluorescent RNA sensor to LARP6.

The term “contacting” refers to bringing the test compound into direct physical contact with the first and/or second fluorescent compound. Contacting may be achieved in many different ways such, for example, as by mixing the substances together.

The fluorescence of the first and/or second fluorescent compound may be detected using a fluorescence measurement technique. Examples of fluorescent measurement techniques include fluorometry, microscopy, FRET, and polarization.

The method may be performed using one or both of the first fluorescent compound and second fluorescent compound because both of the fluorescent compounds are sensitive to LARP6 binding and/or binding to the test compound. An increase or decrease in the detected fluorescence may indicate binding of the test compound to the first fluorescent compound and/or the second fluorescent compound. When the test compound binds to the respective fluorescent compound, the structure of the 5′SL changes, causing the fluorophore to move to a different position, which cause its fluorescence intensity to change. Using both of the first and second fluorescent compounds in this method provides a way to validate or confirm the results from testing the other fluorescent compound.

In one example, when the first fluorescent compound has the fluorophore 2AP substituted at the G10 position of the collagen ɑ1(I) 5′ stem loop mRNA, a decrease in the fluorescence intensity indicates the test compound has bound to the first fluorescent compound and, therefore, the test compound may competitively inhibit or interfere with collagen ɑ1(I) 5′ stem loop mRNA binding to LARP6 and may be an antifibrotic drug candidate. When the first fluorescent compound has the fluorophore 2AP substituted at the G10 position of the collagen ɑ1(I) 5′ stem loop mRNA and the fluorescence intensity increases, this indicates the first fluorescent compound has bound to LARP6.

In another example, when the second fluorescent compound has the fluorophore PC at the C37 position of the collagen ɑ2(I) 5′ stem loop mRNA, a decrease in the fluorescence intensity indicates the test compound has bound to the second fluorescent compound and, therefore, the test compound may competitively inhibit or interfere with collagen ɑ2(I) 5′ stem loop mRNA binding to LARP6 and may be an antifibrotic drug candidate. When the second fluorescent compound has the fluorophore PC at the C37 position of the collagen ɑ2(I) 5′ stem loop mRNA and the fluorescence intensity decreases when contacted with LARP6, this indicates binding of the second fluorescent compound to LARP6.

It may be possible to use modified versions of the fluorophore-substituted SEQ ID NO: 1 and 2 where certain nucleic acids are different than the fluorophore-substituted SEQ ID NO: 1 and 2, but the modified versions maintain the LARP6-binding function. Such modified versions may have 90%, 95%, or 99% sequence identity with the unmodified fluorophore-substituted SEQ ID NO: 1 and 2, respectively.

As discussed above, the fluorescent collagen ɑ1(I) and ɑ2(I) mRNA compounds may be used to provide high-throughput drug screening tests to find competitive inhibitors of LARP6 binding. By measuring the fluorescence intensity of the first and/or second fluorescent compounds, test compounds that interact with the LARP6 binding site of the 5′ stem-loop mRNAs can be detected. Such compounds may act as 5′ stem-loop blockers or competitors to the LARP6 recognition of the 5′ stem-loop mRNA, with a potential to be developed into specific antifibrotic drugs.

### EXAMPLE

This example is provided to illustrate particular examples of the first and second fluorescent compounds and the screening method. The details of this example do not limit the scope of possible examples of the first and second fluorescent compounds or the methods in any way.

FIG. 1 is a diagram of the sequence of the collagen ɑ1(I) and ɑ2(I) 5′SL, with the active, LARP6-binding fold circled with dashed lines. The 2AP substitution at position 10 made to create the fluorescent ɑ1(I) 5′SL compound and the PC substitution at position 37 of the ɑ2(I) 5′SL compound are indicated by arrows.

In the fluorescent ɑ1(I) 5′SL compound, the G10 nucleotide of collagen of ɑ1(I) 5′SL was substituted with 2-amino-purine (2AP) and, in the fluorescent ɑ2(I) 5′SL compound, the C37 nucleotide of ɑ2(I) 5′SL was substituted with pyrrolo-cytidine (PC). These substitutions made the mRNAs fluorescent.

The fluorescence intensity of the 2AP and PC fluorophores depends on their environment in the RNA fold, and the environment changes upon LARP6 or test compound binding to the RNA. The increase in fluorescence is seen if a fluorophore rotates outwards from its position within the RNA. This was seen when LARP6 bound to the fluorescent ɑ1(I) 5′SL compound. The fluorescence decreases if a fluorophore is masked by the bound drug or LARP6. This was seen with a drug that bound both sensors and with LARP6 binding to the fluorescent ɑ2(I) 5′SL compound.

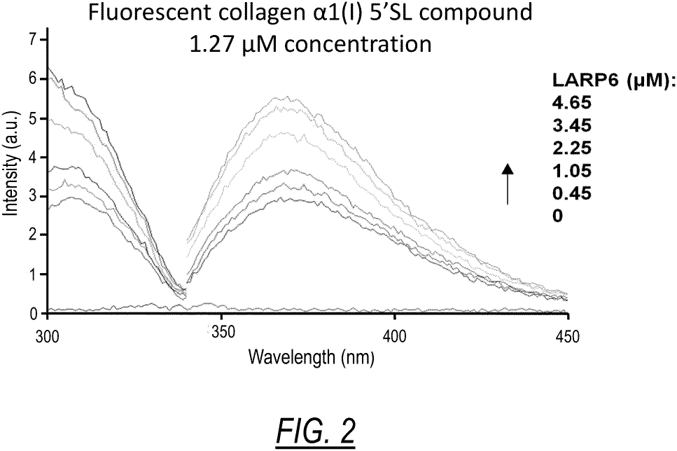

The graph in FIG. 2 shows that the fluorescent ɑ1(I) 5′SL compound responded to LARP6 binding by increasing its fluorescence. The increase resulted from flipping of the 2AP at the G10 position out of the fold. The increase in intensity of the excitation spectrum (left, measured at 370 nM emission) and the emission spectrum (right, measured at 310 nM excitation) were apparent when increasing concentrations of recombinant LA-domain of LARP6 were added to the sensor. The LA-domain is necessary and sufficient for 5′SL RNA recognition by LARP6 and recapitulates the full size LARP6 binding.

The increase in intensity of fluorescence emission with binding of the LA-domain was quantified by measuring the fluorescence of free fluorescent ɑ1(I) 5′SL compound and the same compound bound with saturating amounts of the LA-domain.

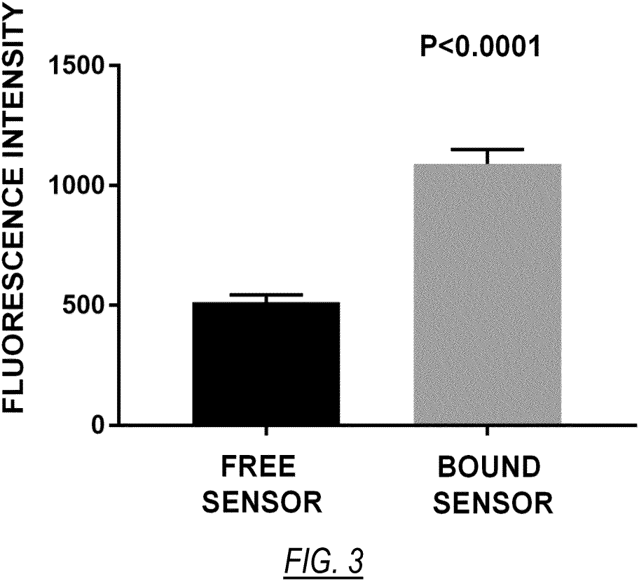

The bar graph of FIG. 3 shows the intensity of fluorescent emission of multiple replicates of the fluorescent ɑ1(I) 5′SL compound alone (black bar) and of the fluorescent ɑ1(I) 5′SL compound bound to the LA-domain (gray bar). The intensity increased about 2-fold with statistical significance of p<0.0001.

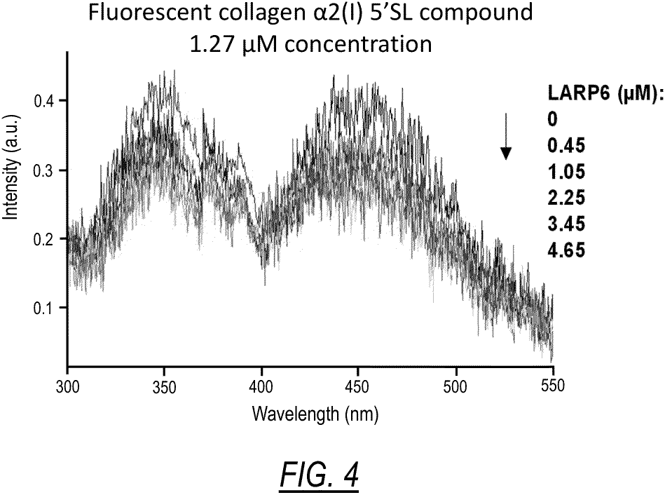

The graph of FIG. 4 shows the results with LA-domain binding to the fluorescent ɑ2(I) 5′SL compound. This compound was less bright because its fluorophore has an emission that is about 7-fold less intense than that of the fluorescent ɑ1(I) 5′SL compound. The fluorescent ɑ2(I) 5′SL compound responded to LA-domain binding by decreasing its fluorescence, indicating that the bound protein shielded the PC fluorophore.

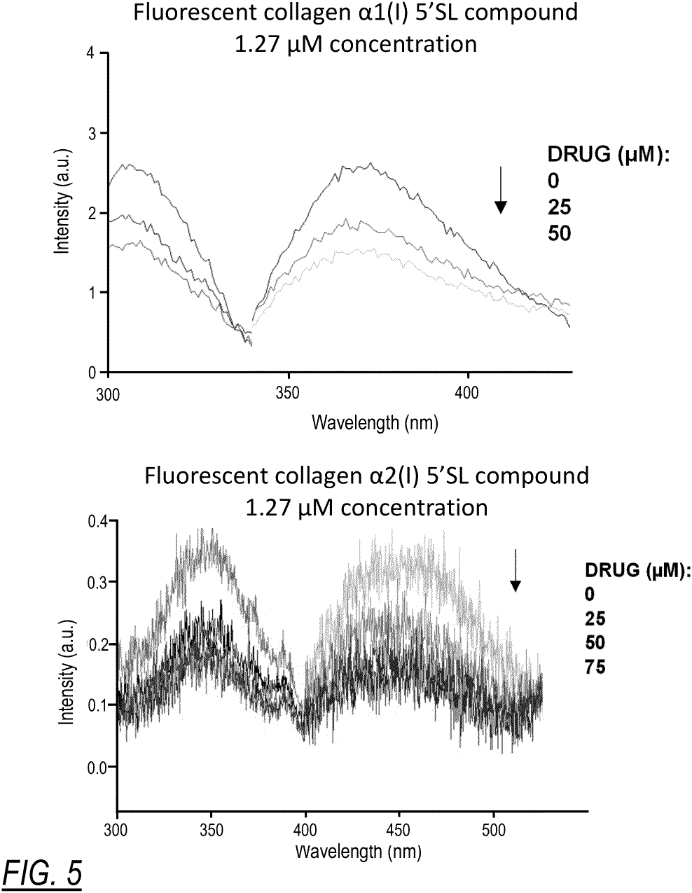

The decrease in the excitation spectrum (left, measured at 450 nM emission) and the emission spectrum (right, measured at 340 nM excitation) with increasing concentrations of recombinant LA-domain is shown in FIG. 5. The relative decrease in fluorescence intensity upon LARP6 binding indicates that this compound is as sensitive as the fluorescent ɑ1(I) 5′SL compound.

Referring to FIG. 5, the fluorescence measurements were performed using epirubicin as the test compound or drug. The data show this test compound decreases the fluorescence of both the fluorescent ɑ1(I) 5′SL compound and the fluorescent ɑ2(I) 5′SL compound. FIG. 5 shows that when this test compound was added in micromolar concentrations to the fluorescent ɑ1(I) 5′SL compound (top panel), the fluorescence intensity decreased in the concentration dependent manner. A similar result was obtained with the fluorescent ɑ2(I) 5′SL compound (bottom panel), which also showed a decrease in fluorescence with increasing drug concentrations.

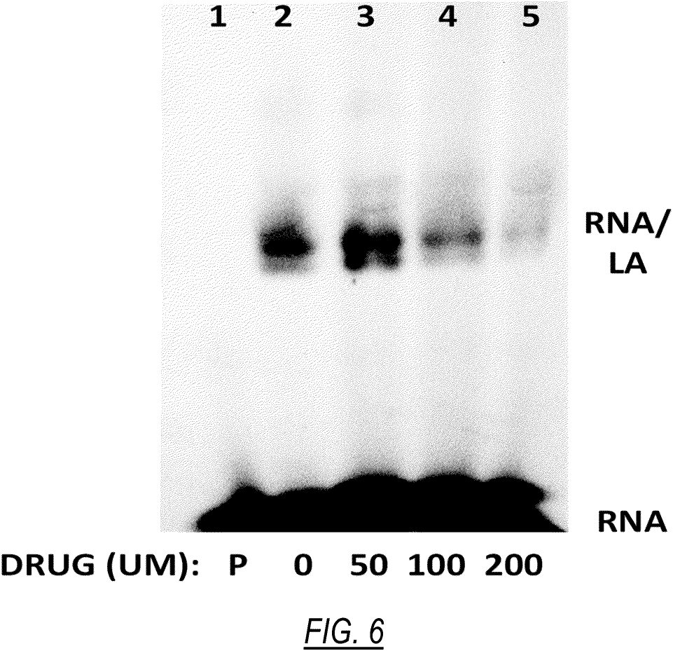

Cross-validation with both fluorescent compounds indicated that the test compound binds in the active site of the 5′SL mRNA and that it may interfere with binding of LARP6. The inhibitory effect of this test compound on LARP6 binding was directly tested by gel mobility shift experiments, the results of which are in FIG. 6. This assay confirms the formation of a protein/RNA complex by physically separating them from free RNA. Without the test compound, the recombinant LA-domain of LARP6 formed a complex with 5′SL RNA (FIG. 6, lane 2, RNA/LA). The formation of RNA/LA complex was diminished with the concentrations of test compound >50 µM (lanes 4 and 5), indicating that the test compound could compete with the LA-domain for binding to 5′SL RNA. This proves that the fluorescent 5′SL RNA compounds can be used to identify molecules that interact with 5′SL RNA and that are competitors of LARP6 binding and may be antifibrotic drugs.

The compositions and methods are not limited to the details described in connection with the example embodiments. There are numerous variations and modification of the compositions and methods that may be made without departing from the scope of what is claimed.

That which is claimed is:

1. A composition comprising a first collagen mRNA nucleotide sequence having at least 90% sequence identity with SEQ ID NO: 1 and a binding affinity for LARP6, the first collagen mRNA nucleotide sequence having at least one fluorophore located at nucleic acid position G10 and/or C38 of SEQ ID NO: 1.

2. The composition of claim 1, wherein the first collagen mRNA nucleotide sequence is SEQ ID NO: 1 having at least one fluorophore located at nucleic acid position G10 and/or C38 of SEQ ID NO: 1.

3. The composition of claim 1, wherein the at least one fluorophore located at nucleic acid position G10 and/or C38 of SEQ ID NO: 1 is substituted at position G10 and/or C38 of SEQ ID NO: 1.

4. The composition of claim 1, wherein the at least one fluorophore located at nucleic acid position G10 and/or C38 of SEQ ID NO: 1 includes 2-aminopurine substituted at nucleic acid position G10 and/or pyrrolo-cytidine substituted at nucleic acid position C38.

5. The composition of claim 1, wherein the first collagen mRNA nucleotide sequence is SEQ ID NO: 1 having nucleic acid position G10 substituted for 2-aminopurine.

6. The composition of claim 1, further comprising a second collagen mRNA nucleotide sequence having at least 90% sequence identity with SEQ ID NO: 2 and a binding affinity for LARP6, the second collagen mRNA nucleotide sequence having at least one fluorophore located at nucleic acid position G9 and/or C37 of SEQ ID NO: 2.

7. The composition of claim 6, wherein the second collagen mRNA nucleotide sequence is SEQ ID NO: 2 having at least one fluorophore located at nucleic acid position G9 and/or C37 of SEQ ID NO: 2.

8. The composition of claim 6, wherein the at least one fluorophore located at nucleic acid position nucleic acid position G9 and/or C37 of SEQ ID NO: 2 is substituted at position G9 and/or C37 of SEQ ID NO: 2.

9. The composition of claim 6, wherein the at least one fluorophore located at nucleic acid position nucleic acid position G9 and/or C37 of SEQ ID NO: 2 includes 2-aminopurine substituted at nucleic acid position G9 and/or pyrrolo-cytidine substituted at nucleic acid position C37.

10. The composition of claim 6, wherein the second collagen mRNA nucleotide sequence is SEQ ID NO: 2 having nucleic acid position C37 substituted for pyrrolo-cytidine.

11. A composition comprising a first collagen mRNA nucleotide sequence having at least 90% sequence identity with SEQ ID NO: 2 and a binding affinity for LARP6, the first collagen mRNA nucleotide sequence having at least one fluorophore located at nucleic acid position G9 and/or C37 of SEQ ID NO: 2.

12. The composition of claim 11, wherein the first collagen mRNA nucleotide sequence is SEQ ID NO: 2 having at least one fluorophore located at nucleic acid position G9 and/or C37 of SEQ ID NO: 2.

13. The composition of claim 11, wherein the at least one fluorophore located at nucleic acid position G9 and/or C37 of SEQ ID NO: 2 is substituted at position G9 and/or C37 of SEQ ID NO: 2.

14. The composition of claim 11, wherein the at least one fluorophore located at nucleic acid position nucleic acid position G9 and/or C37 of SEQ ID NO: 2 includes 2-aminopurine substituted at nucleic acid position G9 and/or pyrrolo-cytidine substituted at nucleic acid position C37.

15. The composition of claim 11, wherein the first collagen mRNA nucleotide sequence is SEQ ID NO: 2 having nucleic acid position C37 substituted for pyrrolo-cytidine.

16. The composition of claim 11, further comprising a second collagen mRNA nucleotide sequence having at least 90% sequence identity with SEQ ID NO: 1 and a binding affinity for LARP6, the second collagen mRNA nucleotide sequence having at least one fluorophore located at nucleic acid position G10 and/or C38 of SEQ ID NO: 1.

17. The composition of claim 16, wherein the second collagen mRNA nucleotide sequence is SEQ ID NO: 1 having at least one fluorophore located at nucleic acid position G10 and/or C38 of SEQ ID NO: 1.

18. The composition of claim 16, wherein the at least one fluorophore located at nucleic acid position G10 and/or C38 of SEQ ID NO: 1 is substituted at position G10 and/or C38 of SEQ ID NO: 1.

19. The composition of claim 16, wherein the at least one fluorophore located at nucleic acid position G10 and/or C38 of SEQ ID NO: 1 includes 2-aminopurine substituted at nucleic acid position G10 and/or pyrrolo-cytidine substituted at nucleic acid position C38.

20. The composition of claim 16, wherein the second collagen mRNA nucleotide sequence is SEQ ID NO: 1 having nucleic acid position G10 substituted for 2-aminopurine.

21. A method comprising:
detecting whether a test compound affects binding of a fluorescent RNA sensor to LARP6 by contacting the fluorescent RNA sensor with the test compound;
the fluorescent RNA sensor comprising a first collagen mRNA nucleotide sequence having at least 90% sequence identity with SEQ ID NO: 1 and a binding affinity for LARP6, the first collagen mRNA nucleotide sequence having at least one fluorophore located at nucleic acid position G10 and/or C38 of SEQ ID NO: 1.

22. The method of claim 21, wherein the first collagen mRNA nucleotide sequence is SEQ ID NO: 1 having at least one fluorophore located at nucleic acid position G10 and/or C38 of SEQ ID NO: 1.

23. The method of claim 21, wherein the at least one fluorophore located at nucleic acid position G10 and/or C38 of SEQ ID NO: 1 is substituted at position G10 and/or C38 of SEQ ID NO: 1.

24. The method of claim 21, wherein the at least one fluorophore located at nucleic acid position G10 and/or C38 of SEQ ID NO: 1 includes 2-aminopurine substituted at nucleic acid position G10 and/or pyrrolo-cytidine substituted at nucleic acid position C38.

25. The method of claim 21, wherein the first collagen mRNA nucleotide sequence is SEQ ID NO: 1 having nucleic acid position G10 substituted for 2-aminopurine.

26. The method of claim 21, the fluorescent RNA sensor further comprising a second collagen mRNA nucleotide sequence having at least 90% sequence identity with SEQ ID NO: 2 and a binding affinity for LARP6, the second collagen mRNA nucleotide sequence having at least one fluorophore located at nucleic acid position G9 and/or C37 of SEQ ID NO: 2.

27. The method of claim 26, wherein the second collagen mRNA nucleotide sequence is SEQ ID NO: 2 having at least one fluorophore located at nucleic acid position G9 and/or C37 of SEQ ID NO: 2.

28. The method of claim 26, wherein the at least one fluorophore located at nucleic acid position nucleic acid position G9 and/or C37 of SEQ ID NO: 2 is substituted at position G9 and/or C37 of SEQ ID NO: 2.

29. The method of claim 26, wherein the at least one fluorophore located at nucleic acid position nucleic acid position G9 and/or C37 of SEQ ID NO: 2 includes 2-aminopurine substituted at nucleic acid position G9 and/or pyrrolo-cytidine substituted at nucleic acid position C37.

30. The method of claim 26, wherein the second collagen mRNA nucleotide sequence is SEQ ID NO: 2 having nucleic acid position C37 substituted for pyrrolo-cytidine.

31. A method comprising:
detecting whether a test compound affects binding of a fluorescent RNA sensor to LARP6 by contacting the fluorescent RNA sensor with the test compound;
the fluorescent RNA sensor a first collagen mRNA nucleotide sequence having at least 90% sequence identity with SEQ ID NO: 2 and a binding affinity for LARP6, the first collagen mRNA nucleotide sequence having at least one fluorophore located at nucleic acid position G9 and/or C37 of SEQ ID NO: 2.

32. The method of claim 31, wherein the first collagen mRNA nucleotide sequence is SEQ ID NO: 2 having at least one fluorophore located at nucleic acid position G9 and/or C37 of SEQ ID NO: 2.

33. The method of claim 31, wherein the at least one fluorophore located at nucleic acid position G9 and/or C37 of SEQ ID NO: 2 is substituted at position G9 and/or C37 of SEQ ID NO: 2.

34. The method of claim 31, wherein the at least one fluorophore located at nucleic acid position nucleic acid position G9 and/or C37 of SEQ ID NO: 2 includes 2-aminopurine substituted at nucleic acid position G9 and/or pyrrolo-cytidine substituted at nucleic acid position C37.

35. The method of claim 31, wherein the first collagen mRNA nucleotide sequence is SEQ ID NO: 2 having nucleic acid position C37 substituted for pyrrolo cytidine.

36. The method of claim 31, further comprising a second collagen mRNA nucleotide sequence having at least 90% sequence identity with SEQ ID NO: 1 and a binding affinity for LARP6, the second collagen mRNA nucleotide sequence having at least one fluorophore located at nucleic acid position G10 and/or C38 of SEQ ID NO: 1.

37. The method of claim 36, wherein the second collagen mRNA nucleotide sequence is SEQ ID NO: 1 having at least one fluorophore located at nucleic acid position G10 and/or C38 of SEQ ID NO: 1.

38. The method of claim 36, wherein the at least one fluorophore located at nucleic acid position G10 and/or C38 of SEQ ID NO: 1 is substituted at position G10 and/or C38 of SEQ ID NO: 1.

39. The method of claim 36, wherein the at least one fluorophore located at nucleic acid position G10 and/or C38 of SEQ ID NO: 1 includes 2-aminopurine substituted at nucleic acid position G10 and/or pyrrolo-cytidine substituted at nucleic acid position C38.

40. The method of claim 36, wherein the second collagen mRNA nucleotide sequence is SEQ ID NO: 1 having nucleic acid position G10 substituted for 2-aminopurine.
